(19) United States
(12) Patent Application Publication
(10) Pub. No.: US 2014/0235925 A1
(43) Pub. Date: Aug. 21, 2014

(54) SYSTEMS AND METHODS FOR PORTATIVE MAGNETO THERAPY

(71) Applicant: HIRAM Abi PAZ, Hialeah Gardens, FL (US)

(72) Inventor: HIRAM Abi PAZ, Hialeah Gardens, FL (US)

(21) Appl. No.: 13/771,455

(22) Filed: Feb. 20, 2013

(51) Int. Cl. A61N 2/06; A01G 7/00

(52) U.S. Cl. CPC A61N 2/06; A01G 7/00
USPC 600/9; 47/1.1 R

(57) ABSTRACT

In accordance with one embodiment, a system for applying magnetic fields to one or more objects is provided. The system may include a one or more neodymium disc magnets for providing magnetic fields. In one embodiment, the system may have up to two neodymium disc magnets. For example, the neodymium disc magnets may be up to 1.5 inches in diameter and up to 0.5 inch thick. For example, the neodymium disc magnets may have strength up to 14500 Gauss. The magnetic fields generated are applied to one or more objects. For example the objects may be a one of a human body, animal body, or plants.

### CROSS-REFERENCE TO RELATED APPLICATIONS

This application claims the benefit of U.S. Provisional Patent Application 61/601,450 having a filing date of Feb. 21, 2012, the, entire contents of which are hereby incorporated by reference.

### BACKGROUND OF THE INVENTION

1. Field of the Invention

Embodiments of the present invention generally relate to therapeutic devices, and more particularly to alternate therapeutic devices which utilize magnetic fields.

2. Description of the Related Art

Magnetic fields have been employed for a variety of therapeutic purposes, including relief of pain and inflammation from soft tissue injury and musculoskeletal injury, relaxation of muscles, relief of headaches, treatment of internal organs and a variety of other therapeutic applications. Additionally, magnetic fields have been utilized to enhance and control the timing of plant growth. Magnetic fields are known to increase circulation and blood flow in the tissues subjected to the magnetic field.

Magnetic fields have also proven effective in treatment of arthritis and have also been claimed to dissolve calciferous plaques and deposits in the bodies of humans and animals. Relative movement between the tissue and magnetic field has been shown to cause increased electron flow (eddy current generation) through the tissue and is also associated with the activation of capillary blood flow and relaxation of muscle.

Research has demonstrated the superiority of a dynamic magnetic field having temporally varying magnetic flux density for enhanced activation of capillary blood flow. Microscopic examination of rat mesentery subjected to dynamic magnet fields has shown an increase of capillary activation in response to an oscillating magnetic field developed by oscillation of a permanent magnet in proximity to the subject tissue, whereas no such effect was observed from placement of a static magnet field in the same proximity to such tissue. Beneficial effects have been observed utilizing magnets having field strengths from less than one Hertz to over 500 Hertz with no defined cutoff threshold identified in response to increased frequencies. Additionally, relaxation of muscle tissue has been noted when a magnetic field, oscillating at 80 Hertz, interacts with such tissue. Moving magnetic fields are also reported to have a pain-reducing effect on arthritis joint pain.

A further consideration in magnetic therapy devices is the selection of north pole versus south pole fields for therapeutic applications. Numerous books have been published by Albert Davis, William Philpott M.D. and others proclaiming differentiated effects between north and south pole magnetic fields as disclosed in U.S. Pat. No. 5,389,981 to Riach. Although magnetic fields are a continuous phenomena, there are subtle differences between north pole (or divergent) fields, south pole (or convergent) fields and mixed fields containing both divergent and convergent flux patterns. The benefits of enhanced performance of dynamic fields may be achieved while preserving the orientation of polarity toward the individual by employing magnets in arrays that maintain pole orientation toward the individual while incorporating movement. It is frequently desirable to maintain field orientation to deliver specific therapeutic applications with north-pole or south-pole fields directed at the individual while, at the same time, moving the magnet sources relative to the individual (permanent magnets or DC electromagnets of specified pole orientation).

The resulting varying field will produce the intensified response of a dynamic field while maintaining the polar preference of the individual. It should be noted that the spacing between magnet sources and distance between the magnet and individual will determine if the subject will experience return path flux of the opposite polarity between magnet sources. If a pure field of either pole is required, the magnetic elements can be positioned either closer to each other on a moving array or the array can be moved farther from the subject exactly as would be practiced with a static array to avoid field reversal in the void between discrete magnetic elements.

It is preferable to utilize permanent magnets to provide the magnetic fields as relatively powerful fields can be provided with no energy input required to generate the field. However, special circumstances could confer an advantage on electromagnet sources. DC electromagnets would provide the benefit of maintaining specified pole orientation. The physical movement of an AC electromagnet relative to the individual could effectively increase the kinetic coupling of the device to tissue. Typical prior applications have placed permanent magnets within devices which are moved over affected target tissues or secured to the user so that the magnet does not move relative to the user.

U.S. Pat. No. 5,226,020 to Li et al. discloses a wristwatch having a magnetic body which is proximate to the skin of the user when the wristwatch is worn. A magnetic isolating piece is disposed between the time-keeping mechanism and the magnetic body so as to prevent the magnetic body from interfering with the operation of the time-keeping piece. Although the wristwatch disclosed by Li et al. incorporates a magnet placed proximate to the skin of the user, the magnetic body remains stationary with respect to the wearer, and does not subject the wearer to a moving magnetic field. U.S. Pat. No. 5,389,981 to Riach Jr. discloses eyeglasses having magnets attached thereto so as to subject the eyes to magnetic energy. The magnets are positioned on the eyeglasses to project a north magnetic field into the surrounding area of the eyes to improve blood circulation. The device disclosed by Riach Jr. includes stationary magnets and is intended to project a stationary magnetic field over the eyes.

U.S. Pat. No. 5,295,494 to Rodriguez discloses a housing having a magnet securely positioned therein, the housing configured to accept a band to secure the housing to a user. The configuration of the housing prevents movement of the magnet within the interior of the housing. Thus, the wearer is subjected to a stationary rather than a moving magnetic field. The device disclosed by Onishi in U.S. Pat. No. 4,850,340 likewise utilizes hollow cases containing a magnet, each case configured so as to be pivotably linked to other cases. The magnetic field generator contained within each case is fixed in a stationary position within the case. Thus, the wearer of the Onishi device is subjected to a stationary rather than a moving magnetic field.

U.S. Pat. No. 4,177,796 to Franco- Vila discloses a device for the treatment of arthritis which utilizes a pair of magnets, one magnet affixed to each side of an elastic band which is placed across the end of a housing containing an electromagnet. The paired magnets are caused to vibrate by application of a low frequency alternating magnetic field which causes the magnets and the elastic band to vibrate, so that, upon placing the end of the housing next to the injured tissue, vibration of the magnets acts to massage the tissue. The electromagnet subjects the tissue to be treated to a magnetic field, while the vibrating elastic band subjects the tissue to vibration. U.S. Pat. No. 5,027,795 to Kato et al. discloses a massage machine to be utilized in conjunction with a seat in a vehicle. The device is configured to dispose magnets between the person in the seat and the seat, the magnets being non-moveably affixed to the frame which is positioned on the seat of the vehicle. The magnets of the Kato device are not moved with respect to the user.

U.S. Pat. No. 4,682,584 to Pose discloses a dental care instrument for treating emissions of foreign bodies within the mouth, the instrument having, at its operative end, a magnet which is placed within a patient's mouth. The end of the dental instrument is moved relative to the teeth, thus moving the magnet relative to the teeth. While in some embodiments the magnet is affixed to the end of the dental instrument so that it is rotatable about the end of the dental instrument, alternate embodiments provide a magnet fixed in a stationary position. While the Pose device does provide a relatively simple mechanism for applying a magnetic field to an area to be treated, the user or individual providing treatment must move the dental care instrument with respect to the area to be treated to achieve movement of the magnet.

Likewise, U.S. Pat. No. 4,744,350 to Sato and U.S. Pat. No. 5,382,222 to Yih-Jong both disclose massaging devices having magnets embedded therein, each device being movable over the area to be treated. U.S. Pat. No. 5,323,499 to Chan discloses a mattress including a layer of alternating wooden beads and magnets, the magnets providing a static magnetic field on which the user may rest, the alternating magnets and beads applying a massaging action to the user. The mattress magnets apply a stationary magnetic field to the individual as the individual is sleeping upon the mattress. Permanent magnets may be inertially mounted to the body of an individual so that the movement of the host will create relative movement of the magnet and the target tissue. Permanent or electrical magnets may be used. The inertial mount can be applied to surrounding environments through which the individual moves, such as a truck, desk or stationary support elements of a swing whereby the individual moves through the therapeutic magnetic field.

The inertial mounting system will increase the effectiveness of interaction between individual and the magnetic field by moving the magnetic source relative to the individual. Relative movement between the individual and magnet is provided by the natural movement of the individual, which may additionally cause movement of the magnet. The magnets may be mounted, for example, in the supporting frame of a porch swing so that the swinging motion of the individual in the chair portion of the swing causes relative motion between the magnet and individual.

Such systems provide a changing magnetic field relative to the individual which may help to reduce muscle stiffness and discomfort for persons confined to one position for extended periods such as truck drivers and persons confined to a bed or a chair.

The present invention combines the benefits of portability associated with easily applied permanent magnetic devices with moving magnetic fields relative to tissue. The present invention improves upon prior art biomagnetic devices by providing enhanced relative movement between the magnet and the subject tissue by physically moving the magnetic source. The present invention provides a means for the magnet to be moved relative to the subject tissue in response to inertial or other mechanical energy inputs incidental to the activity of the individual.

### SUMMARY

In accordance with one embodiment, a system for applying magnetic fields to one or more objects is provided. The system may include a one or more neodymium disc magnets for providing magnetic fields. In one embodiment, the system may have up to two neodymium disc magnets. For example, the neodymium disc magnets may be up to 1.5 inches in diameter and up to 0.5 inch thick. For example, the neodymium disc magnets may have strength up to 14500 Gauss. The magnetic fields generated are applied to one or more objects. For example the objects may be a one of a human body, animal body, or plants.

The system may also have one or more magnet holders for secure one or more neodymium disc magnets. For example the magnet holders may be wooden holders. Optionally, the magnet holders are composed of plastic, composite, carbon fiber, or the like or any combination thereof.

One or more electrical motors attached to the magnet holders. As the electrical motors rotate the magnets rotate as well via the magnet holders. The rotating motor creates a sinusoidal magnetic field. For example, the motor is a six volts direct current electric motor. In one embodiment, one or more screws may be used for fastening the magnet holders to the electric motor.

A potentiometer may also be provided for controlling the electrical motors. For example, the potentiometers are at least 10 kilo ohms or up to 10 kilo ohms potentiometers. The potentiometer may be configured to alter the electrical motors speed to generate sinusoidal frequencies up to thirty hertz. In one embodiment, the potentiometer may be configured to generate sinusoidal frequency of fifteen hertz. The fifteen hertz frequency can be used to treat damaged articulate cartilages of the joints. In one embodiment, the potentiometer may be configured to generate sinusoidal frequency of between twenty five hertz to thirty hertz. The twenty five hertz to thirty hertz frequency may be used to stimulate fibroblasts, increasing production of collagen fibers.

For example, the fifteen hertz frequency may be used to treat gout. In another option, the potentiometer may be configured to generate sinusoidal frequency of seven hertz, applying the seven hertz frequency to treat the object for stress. In an alternate embodiment, potentiometer may be configured to generate sinusoidal frequency of three hertz. The three hertz frequency may be used to treat the objects for sleep disorders.

The neodymium disc magnets, magnet holders, electrical motors, and the one or more potentiometer may all be enclosed within housing. The housing is small so as to allow the system to be a portative magneto therapy system. For example, the housing is gun shaped where the gun trigger acts as a on or off button. In one embodiment, the housing may have a cylindrical compartment for enclosing at least the one or more neodymium disc magnets, the one or more magnet holders, the one or more electrical motors. The cylindrical compartment may be up to 4.5 inches in diameter and 7 inches in length. The housing may have mechanism to choose different frequency setting for the system. For example, the mechanism may be a dial know electrically coupled to the potentiometer to switch between different settings. Optionally, the housing may also have indicator a one or more indicators for indicating the different state of the system. For example, the housing may be one of a plastic, wood, composite, carbon fiber, or the like or any combination thereof.

### BRIEF DESCRIPTION OF THE DRAWINGS

The drawings, in which like numerals represent similar parts, illustrate generally, by way of example, but not by way of limitation, various embodiments discussed in the present document.

FIG. 1 illustrates a portative magneto therapy system in accordance with an embodiment.

FIG. 2 illustrates internal component of the portative magneto therapy system in accordance with an embodiment.

FIG. 3 illustrates exploded view of the portative magneto therapy system in accordance with an embodiment.

### DETAILED DESCRIPTION

The foregoing summary, as well as the following detailed description of certain embodiments of the subject matter set forth herein, will be better understood when read in conjunction with the appended drawings. As used herein, an element or step recited in the singular and proceeded with the word “a” or “an” should be understood as not excluding plural of said elements or steps, unless such exclusion is explicitly stated. Furthermore, references to “one embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

To the extent that the term “includes” is used in either the detailed description or the claims, such term is intended to be inclusive in a manner similar to the term “comprising” as “comprising” is interpreted when employed as a transitional word in a claim. Furthermore, the term “or” as used in either the detailed description or the claims is intended to mean an inclusive “or” rather than an exclusive “or.” That is, unless specified otherwise, or clear from the context, the phrase “X employs A or B” is intended to mean any of the natural inclusive permutations. That is, the phrase “X employs A or B” is satisfied by any of the following instances: X employs A; X employs B; or X employs both A and B.

In the following detailed description, reference is made to the accompanying drawings which form a part hereof, and in which are shown by way of illustration specific embodiments in which the subject matter disclosed herein may be practiced. These embodiments, which are also referred to herein as “examples,” are described in sufficient detail to enable those skilled in the art to practice the subject matter disclosed herein. It is to be understood that the embodiments may be combined or that other embodiments may be utilized, and that structural, logical, and electrical variations may be made without departing from the scope of the subject matter disclosed herein. The following detailed description is, therefore, not to be taken in a limiting sense, and the scope of the subject matter disclosed herein is defined by the appended claims and their equivalents.

In the description that follows, like numerals or reference designators will be used to refer to like parts or elements throughout. In this document, the terms “a” or “an” are used, as is common in patent documents, to include one or more than one. In this document, the term “or” is used to refer to a nonexclusive or, unless otherwise indicated. Furthermore, references to “one embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property. The terms “patient” and “object”, as used herein, mean and include humans, or animals, or plants.

Crisis of gout frequently is unresponsive; to drugs. On the other hand some traumas can cause pains and bruises which take several days to disappear. Finally psychological stress causes many undesirable effects like sleep disorders, migraines difficult to treat with medications. The primary physician frequently deals with these situations in his office. A portative magneto-therapy machine described herein provides treatment of gout, different types of traumas, migraine and psychological stress at the primary physician's office.

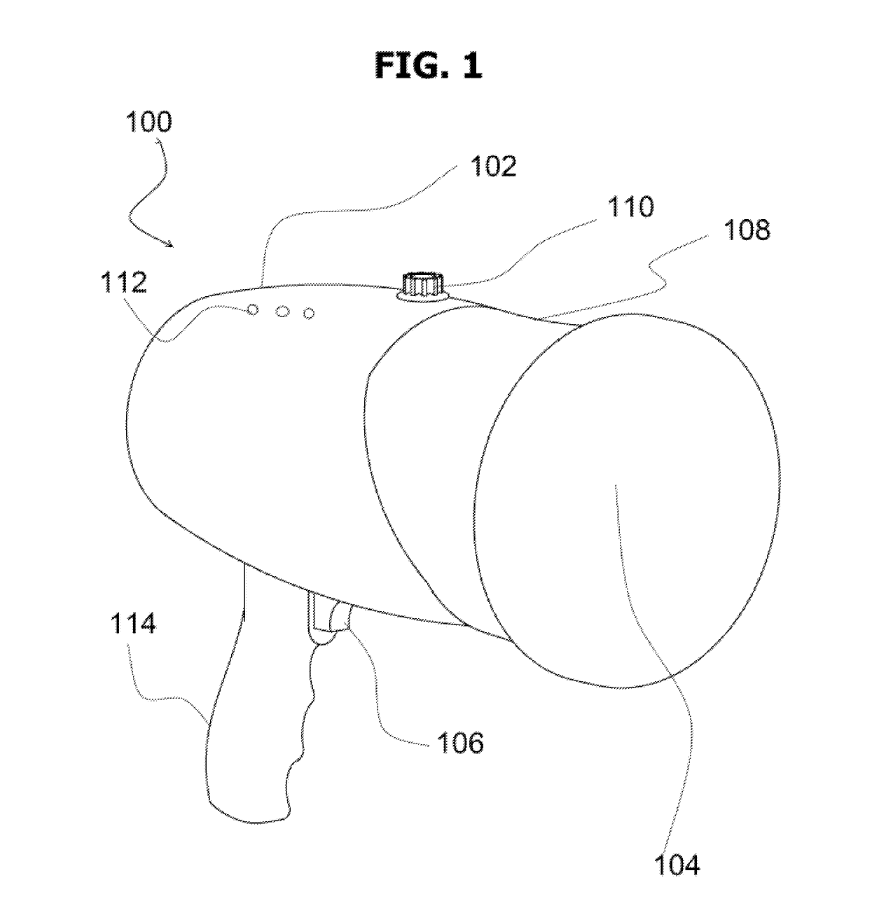

FIG. 1 illustrates a portative magneto therapy system 100 in accordance with an embodiment. The portative magneto therapy system 100 generates low frequency magnetic fields. FIG. 1 illustrates the system 100 housing 102 having a front face 104. The front face 104 is applies the magnetic fields generated to one or more objects. For example the objects may be a one of a human body, animal body, or plants.

The housing 102 is small so as to allow the system to be a portative magneto therapy system 100. The housing 102 is gun shaped where the gun trigger 106 acts as a on or off button. In one embodiment, the housing 102 may have a cylindrical compartment 108 for enclosing at least one or more magnets, one or more magnet holders, one or more electrical motors. The cylindrical compartment 108 may be up to 4.5 inches in diameter and 7 inches in length.

The housing 102 may have mechanism to choose different frequency setting for the system. For example, the mechanism may be a dial 110 electrically coupled to a potentiometer (not shown) to switch between different frequency settings. Optionally, the housing 102 may also have one or more indicators 112 for indicating the different state of the system 100. For example, the housing 102 may be one of a plastic, wood, composite, carbon fiber, or the like or any combination thereof. The housing may also have a handle 114 allowing a user of the system 100 to hold the portative magneto therapy system 100.

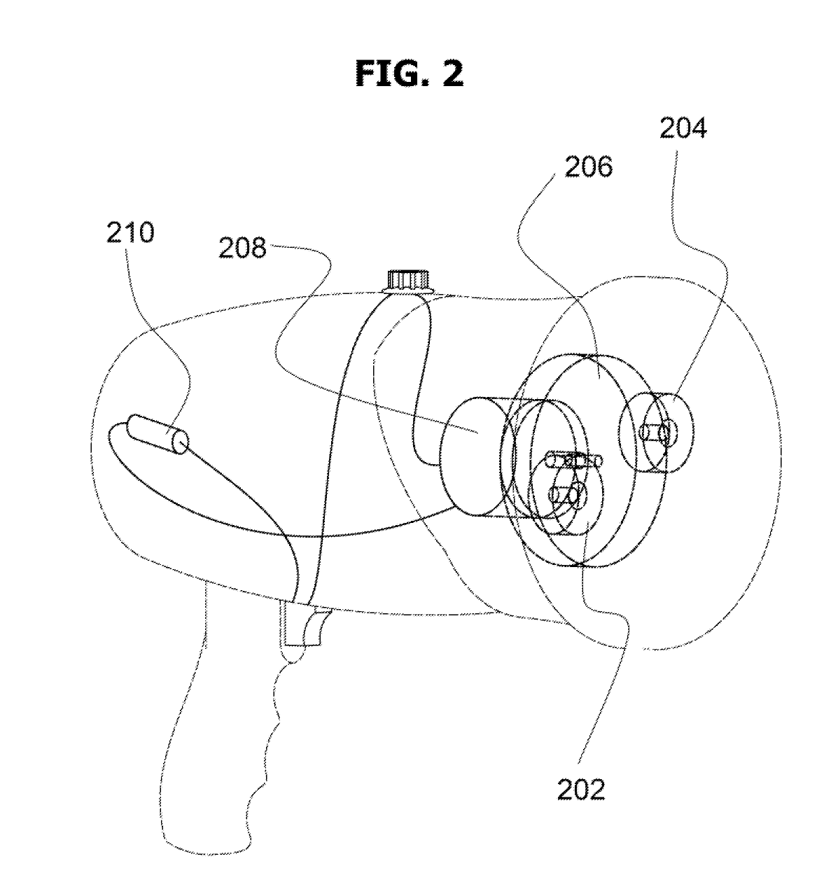

FIG. 2 illustrates internal component of the portative magneto therapy system 100 in accordance with an embodiment. As shown in FIG. 2, the system may have two neodymium disc magnets (202, 204) for providing magnetic fields. For example, the neodymium disc magnets (202, 204) may be up to 1.5 inches in diameter and up to 0.5 inch thick. For example, the neodymium disc magnets may have strength up to 14500 Gauss. The magnetic fields generated are applied to one or more objects. For example the objects may be a one of a human body, animal body, or plants.

FIG. 2 also shows a circular magnet holder 206 having two slots for securing two neodymium disc magnets (202, 204). For example the magnet holder 206 may be wooden holders. Optionally, the magnet holder 206 is composed of plastic, composite, carbon fiber, or the like or any combination thereof. The system 100 also has an electrical motor 208 attached to the magnet holders 206. In an exemplary embodiment, the electrical motor 208 is battery operated. Once the electrical motor 208 is turned on the front face of the housing is pressed against the objects. For example, the front face may be pressed against the objects for two or three minutes.

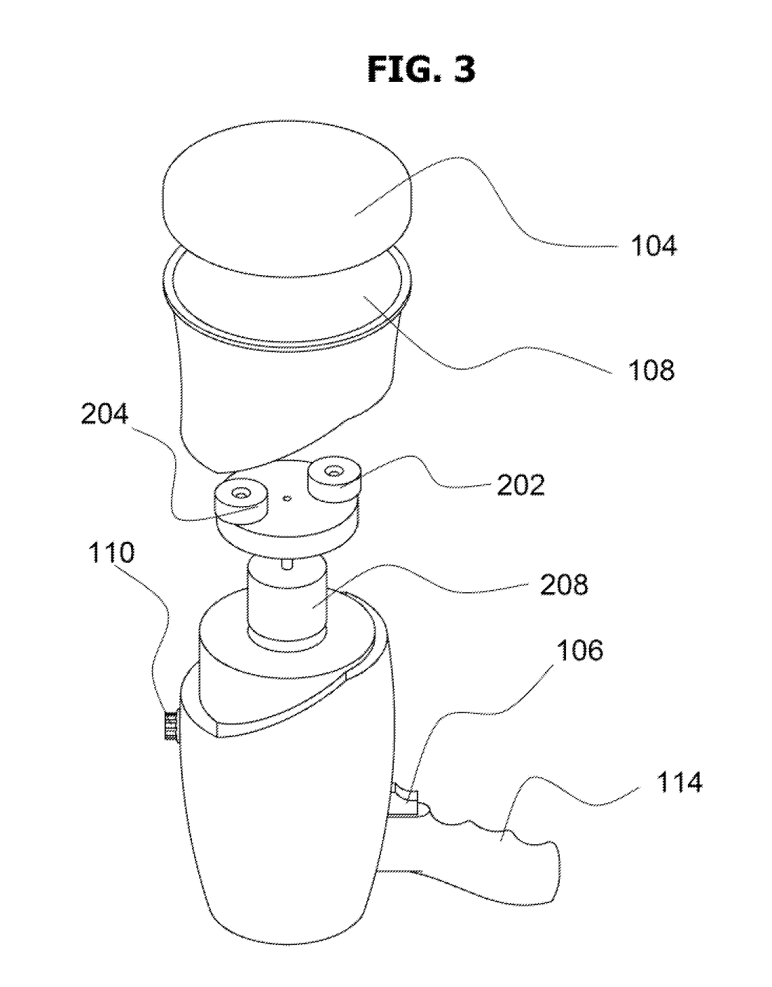

As the electrical motor 208 rotates the magnets (202, 204) rotate as well via the magnet holders 206. The rotating motor 208 creates a sinusoidal magnetic field. For example, the motor 208 may be a six volts direct current electric motor. In one embodiment, one or more screws may be used for fastening the magnet holders 206 to the electric motor 208. Furthermore, the magnets (202, 204) may be fastened to the magnet holder 206 using screws. FIG. 3 illustrates exploded view of the portative magneto therapy system in accordance with an embodiment.

Additionally, a potentiometer 210 may also be provided for controlling the electrical motor 208. For example, at least 10 kilo ohms or up to 10 kilo ohms potentiometer 210. The potentiometer 210 may be configured to alter the electrical motors speed to generate sinusoidal frequencies up to thirty hertz.

The motor 208 makes the magnets rotate at a speed or revolution per minute generating a sinusoidal magnetic field. For example, the rotation of the motor may be graduated in a range from O to 3O HZ. The 10 HZ frequency may be used to relief the effects of traumas. Magnets (204, 202) must be oriented one facing the north and the other one facing the south poles.

In one embodiment, the potentiometer 210 may be configured to generate sinusoidal frequency of fifteen hertz. The fifteen hertz frequency can be used to treat the objects with gout. In another option, the potentiometer 210 may be configured to generate sinusoidal frequency of seven hertz, applying the seven hertz frequency to treat the object for stress. In an alternate embodiment, potentiometer 210 may be configured to generate sinusoidal frequency of three hertz. The three hertz frequency may be used to treat the objects for sleep disorders.

In addition to the potentiometer 210, the system 100 may also have a processor for processing instruction set. It is to be understood that the embodiments and features that are described herein may be implemented by hardware, software, firmware or any combination thereof. Various embodiments described herein are described in the general context of methods or processes, which may be implemented in one embodiment by a computer program product, embodied in a computer-readable medium, including computer-executable instructions, such as program code, executed by computers in networked environments.

The various embodiments and/or components, for example, the modules, elements, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as an optical disk drive, solid state disk drive (e.g., flash RAM), and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), application specific integrated circuits (ASICs), field-programmable gate arrays (FPGAs), graphical processing units (CPUs), logic circuits, and any other circuit or processor capable of executing the functions described herein. The above examples are exemplary only, and are thus not intended to limit in any way the definition and/or meaning of the term “computer”.

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the invention. The set of instructions may be in the form of a software program, which may form part of a tangible non-transitory computer readable medium or media. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, the terms “software”, “firmware” and “algorithm” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions, types of materials and coatings described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. §112, sixth paragraph, unless and until such claim limitations expressly use the phrase “means for” followed by a statement of function void of further structure.

This written description uses examples to disclose the various embodiments of the invention, including the best mode, and also to enable any person skilled in the art to practice the various embodiments of the invention, including making and using any devices or systems and performing any incorporated methods. The patentable scope of the various embodiments of the invention is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal languages of the claims.

What is claimed is:

1. A system for applying a magnetic fields to plurality of objects, the system comprising:
a plurality of neodymium disc magnets for providing magnetic fields, as applied to a plurality of objects;
a plurality of magnet holders configured to fasten the plurality of neodymium disc magnets within place;
a plurality of electrical motors configured to moving the plurality of neodymium magnets, the plurality of electrical motors attached to each of the plurality of neodymium disc magnets via the plurality of magnet holders;
a plurality of potentiometer configured for controlling the plurality of electrical motors; and
a housing enclosing the plurality of neodymium disc magnets, the plurality of magnet holders, the plurality of electrical motors, and the plurality of potentiometer so as to provide a portative magneto therapy system.

2. The system of claim 1, wherein two neodymium disc magnets are used.

3. The system of claim 1, wherein the plurality of neodymium disc magnets are up to 1.5 inches in diameter and up to 0.5 inch thick.

4. The system of claim 1, wherein the plurality of neodymium disc magnets having strength up to 14500 Gauss.

5. The system of claim 1, wherein plurality of magnet holders are wooden holders.

6. The system of claim 1, wherein plurality of magnet holders are composed of plastic, composite, carbon fiber, or the like or any combination thereof.

7. The system of claim 1, wherein the rotating motor creates a sinusoidal magnetic field.

8. The system of claim 1, wherein the motor is a six volts direct current electric motor.

9. The system of claim 1 further comprises plurality of screws for fastening the plurality of magnet holders to the electric motor.

10. The system of claim 1, wherein the plurality of potentiometers are at least 10 kilo ohms potentiometers.

11. The system of claim 1, wherein the plurality of potentiometers are up to 10 kilo ohms potentiometers.

12. The system of claim 1, wherein potentiometer configured to generate sinusoidal frequencies up to thirty hertz.

13. The system of claim 1, wherein potentiometer configured to generate sinusoidal frequency of fifteen hertz, applying the fifteen hertz frequency to treat damaged articulate cartilages of the joints.

14. The system of claim 1, wherein potentiometer configured to generate sinusoidal frequency of between twenty five hertz to thirty hertz, applying the twenty five hertz to thirty hertz frequency to stimulate fibroblasts, increasing production of collagen fibers.

15. The system of claim 1, wherein potentiometer configured to generate sinusoidal frequency of seven hertz, applying the seven hertz frequency to treat the object with stress.

16. The system of claim 1, wherein potentiometer configured to generate sinusoidal frequency of three hertz, applying the three hertz frequency to treat the object with sleep disorders.

17. The system of claim 1, wherein the housing is gun shaped where the gun trigger acting as a on or off button.

18. The system of claim 1, wherein the housing having a cylindrical compartment for enclosing at least the plurality of neodymium disc magnets, the plurality of magnet holders, the plurality of electrical motors.

19. The system of claim 1, wherein the housing having mechanism to choose different frequency setting for the system and a plurality of indicators for indicating the different state of the system.

20. The system of claim 1, wherein the housing is one of a plastic, wood, composite, carbon fiber, or the like or any combination thereof.
